(19) United States
(12) Patent Application Publication
(10) Pub. No.: US 2007/0009587 A1
(43) Pub. Date: Jan. 11, 2007

(54) Method and device for coating a continuous strip of microprojection members

(76) Inventors: Peter Daddona, Mento Park, CA (US); John Dohner, Portola Valley, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/472,165

(22) Filed: Jun. 20, 2006

Related U.S. Application Data
(60) Provisional application No. 60/692,769, filed on Jun. 21, 2005.

(51) Int. Cl. A61F 13/02; A61L 15/16

(52) U.S. Cl. 424/448; 604/500

(57) ABSTRACT

The present invention provides a device and method for selectively providing multiple applications of an agent formulation on the skin piercing portions of a microprojection member to form an agent-containing coating on the microprojections. The formulation is dried to form a solid coating which contains the agent. A continuous conveyor strip bearing multiple microprojection members is spiral wrapped around a product drum so that each microprojection member is conveyed past a reservoir a plurality of times. The reservoir is configured to transfer an application of agent formulation onto the microprojection member each time it passes the reservoir.

### CROSS REFERENCE TO RELATED APPLICATIONS

This application claims the benefit of U.S. Provisional Application Ser. No. 60/ 692,769, filed Jun. 21, 2005.

### FIELD OF THE PRESENT INVENTION

This invention relates to administering and enhancing transdermal delivery of a biologically active agent across the skin. More particularly, the invention relates to a percutaneous delivery system for administering a biologically active agent through the stratum corneum using skin piercing microprojections that have a dry coating of the biologically active agent. Even more particularly, the invention relates to a method and device wherein the coating is formed by multiple applications of a formulation containing the agent.

### BACKGROUND OF THE INVENTION

Active agents (or drugs) are most conventionally administered either orally or by injection. Unfortunately, many active agent are completely ineffective or have radically reduced efficacy when orally administered, since they either are not absorbed or are adversely affected before entering the bloodstream and thus do not possess the desired activity. On the other hand, the direct injection of the agent intravenously or subcutaneously, while assuring no modification of the agent during administration, is a difficult, inconvenient, painful and uncomfortable procedure that sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, passive transdernal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

There have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

A precisely controlled coating method which can reproducibly coat only the skin-piercing portions of a transdermal delivery device is roller coating as disclosed in U.S. Pat. No. 6,855,372, which is hereby incorporated by reference in its entirety. The noted process generally involves delivering multiple microprojection arrays to a roller that has a thin film of an active agent formulation to transfer a controlled amount of the formulation to the tips of the microprojections.

Despite the advantages presented by utilizing transdermal delivery devices having a coating of a biologically active agent, it can be difficult to achieve uniform coatings having the desired loading of active agent. One way of improving uniformity and loading is to apply the solid coating in a series of applications of agent formulation, allowing the formulation to dry between applications. However, this type of process complicates the manufacturing process. Thus, what has been needed is a system for producing a high volume of coated microprojection members, each having a coating formed by multiple applications of agent formulation.

Accordingly, it is an object of the invention is to provide a method and device for transferring multiple applications of a biologically active agent formulation to a microprojection array to form a solid coating.

It is another object of the invention to provide a method and device to increase the loading and uniformity of a biologically active agent on a transdermal delivery device.

It is another object of the invention is to provide a method and device for applying multiple coatings to a microprojection array using a single reservoir of biologically active agent.

It is another object of the invention is to minimize the space required for a high volume process capable of multiple applications of a biologically active agent formulation to a transdermal delivery device.

It is yet another object of the invention to minimize the amount of biologically active agent formulation necessary to effectively coat a high volume of microprojection arrays.

### SUMMARY OF THE INVENTION

In accordance with the above objects and those that will be mentioned and will become apparent below, the device and method for applying a biologically active agent formulation to a microprojection member having a plurality of stratum corneum-piercing microprojections generally comprises a device having a flexible conveyor strip with the microprojection member mounted thereon, a product drum, wherein the conveyor strip is spirally wrapped around the product drum, and a reservoir of the biologically active agent formulation adapted to cooperate with the product drum to transfer at least one, preferably, two applications of the biologically active agent onto the microprojections as the conveyor strip travels around the product drum.

In one embodiment of the invention, the product drum rotates with the flexible conveyor strip.

In another embodiment of the invention, the product drum is fixed. Preferably, in the noted embodiment, the product drum includes a spiral flight configured to constrain the conveyor strip in a spiral wrap pattern. Also preferably, the product drum has a plurality of air holes disposed between the spiral flight to transmit compressed air and reduce friction between the conveyor strip and the product drum.

In a preferred embodiment of the invention, the reservoir comprises an agent formulation holding surface and wherein the product drum is positioned relative to the holding surface so that the microprojection member is conveyed with the microprojections being immersed at a predetermined level through the agent formulation on the holding surface. More preferably, the agent formulation holding surface comprises an outer surface of a rotatable cylindrically-shaped coating drum adapted to receive a film of the biologically active agent formulation. In the noted embodiment, the coating drum can be partially immersed in a container of the biologically active agent formulation.

The invention also comprises a system for applying a biologically active agent formulation to a microprojection member having a plurality of stratum corneum-piercing microprojections, the system including a flexible conveyor strip with the microprojection member mounted thereon, a product drum, wherein the conveyor strip is spirally wrapped around the product drum, and a reservoir of the biologically active agent formulation adapted to cooperate with the product drum to transfer at least two applications of the biologically active agent onto the microprojections as the conveyor strip travels around the product drum, wherein the conveyor strip has a plurality of the microprojection members mounted thereon.

Preferably, the system further includes a drying chamber having controlled temperature and humidity adapted to receive the conveyor strip carrying the microprojection members after transfer of the biologically active agent formulation to the microprojections.

In accordance with the invention, the system can also includes a source of adhesive backing for laminating to the microprojection members.

In another embodiment of the invention, the system further includes a cutter to cut individual microprojection members from the conveyor strip, such as a laser die cutter.

In yet another embodiment of the invention, the system also includes a plunger for mounting the individual microprojection members on a retainer ring.

The invention also includes a method for applying a biologically active agent formulation to a microprojection member having a plurality of stratum corneum-piercing microprojections comprising the steps of providing a flexible conveyor strip with the microprojection member mounted thereon, spiral wrapping the conveyor strip a plurality of times around a product drum, and conveying the microprojection member past a reservoir of the biologically active agent formulation to transfer at least two applications of the biologically active agent onto the microprojections as the conveyor strip travels around the product drum.

In the noted embodiment, the product drum preferably includes a spiral flight configured to constrain the conveyor strip in a spiral wrap pattern. Also preferably, the product drum has a plurality of air holes on a surface of the product drum between the spiral flight so that compressed air can be delivered through the air holes to reduce friction between the conveyor strip and the product drum.

In another embodiment of the invention, the method includes providing the reservoir with a holding surface positioned relative to the product drum so that the microprojection member is conveyed past the reservoir with the microprojections being immersed at a predetermined level through the agent formulation on the holding surface. Preferably, the holding surface comprises a rotating cylindrically-shaped coating drum upon which a film of the agent formulation can be applied. In a further embodiment, the coating drum can be partially immersed in a container of the agent formulation.

The methods of the invention are adapted to process a plurality of microprojection members mounted on the conveyor strip to allow high volume production of microprojection members having a biologically active agent coating.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection array, in accordance with the invention;

FIG. 2 is a perspective view of the microprojection array of FIG. 1 with a coating deposited onto the microprojections;

FIGS. 3 A-C are views of a system for transferring multiple applications of agent formulation to a plurality of microprojection members, according to the invention; wherein 3A is a schematic view of one embodiment of the system, 3B is a schematic view of an alternative embodiment of the system, and 3C is a cross sectional view of one portion of the system in 3B;

FIG. 4 is a perspective view of a product drum of the invention for receiving a spiral wrap of conveyor strip bearing the microprojection members, according to the invention;

FIG. 5 is a cross sectional view of the product drum shown in FIG. 4; and

FIG. 6 is a side view showing the interaction of a product drum conveying microprojection members past a reservoir of agent formulation, according to the invention.

### DETAILED DESCRIPTION OF THE INVENTION

Before describing the present invention in detail, it is to be understood that this invention is not limited to particularly exemplified materials, formulations, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a biologically active agent” includes two or more such agents; reference to “a microprojection” includes two or more such microprojections and the like.

### DEFINITIONS

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more immunologically active agents may be formulated in the biocompatible coatings of the invention, resulting in co-delivery of different immunologically active agents.

The term “biologically active agent”, as used herein, refers to a composition of matter or mixture containing an active agent or drug, which is pharmacologically effective when administered in a therapeutically effective amount. Examples of such active agents include, without limitation, small molecular weight compounds, polypeptides, proteins, oligonucleotides, nucleic acids and polysaccharides.

Suitable biologically active agents that can be employed in the present invention include, without limitation, growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl] carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

The term “immunologically active agent”, as used herein, refers to a composition of matter or mixture containing an antigenic agent and/or a “vaccine” from any and all sources, which is capable of triggering a beneficial immune response when administered in an immunologically effective amount. Examples of such agents include, without limitation, viruses and bacteria, protein-based vaccines, polysaccharide-based vaccine, and nucleic acid-based vaccines.

Suitable antigenic agents that can be employed in the present invention include, without limitation, antigens in the form of proteins, polysaccharide conjugates, oligosaccharides, and lipoproteins. These subunit vaccines in include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxine subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitidis (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as respiratory syncytial virus (RSV), cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitidis, pseudomonas aeruginosa, streptococcus pneumoniae, treponema pallidum, and vibrio cholerae, and mixtures thereof.

A number of commercially available vaccines, which contain antigenic agents, also have utility with the present invention including, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Immunologically active agents comprising nucleic acids that can be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants, which, together with the antigenic agent, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (IL-1β163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

The noted immunologically active agents can also be in various forms, such as free bases, acids, charged or uncharged molecules, components of molecular complexes or pharmaceutically acceptable salts. Further, simple derivatives of the agents (such as ethers, esters, amides, etc.), which are easily hydrolyzed at body pH, enzymes, etc., can be employed.

The term “biologically effective amount” or “biologically effective rate”, as used herein, refers to the amount or rate of the immunologically active agent needed to stimulate or initiate the desired immunologic, often beneficial result. The amount of the immunologically active agent employed in the coatings of the invention will be that amount necessary to deliver an amount of the immunologically active agent needed to achieve the desired immunological result. In practice, this will vary widely depending upon the particular immunologically active agent being delivered, the site of delivery, and the dissolution and release kinetics for delivery of the immunologically active agent into skin tissues.

As will be appreciated by one having ordinary skill in the art, the dose of the immunologically active agent that is delivered can also be varied or manipulated by altering the microprojection array (or patch) size, density, etc.

The term “coating formulation”, as used herein, is meant to mean and include a freely flowing composition or mixture that is employed to coat the microprojections and/or arrays thereof.

The term “biocompatible coating” and “solid coating”, as used herein, is meant to mean and include a “coating formulation” in a substantially solid state.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human. The microprojections can be formed in different shapes, such as needles, hollow needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “transdermal” means the delivery of an agent (e.g., a drug or vaccine) into and/or through the skin for local or systemic therapy.

The term “transdermal flux” means the rate of transdermal delivery.

Conventional methods for applying agent-containing coatings to microprojections typically involve partially immersing the microprojection(s) in the agent formulation so that only a portion of the entire length of the microprojections is coated. After coating, the microprojections are removed from the coating formulation and then dried to form a dry agent-containing coating on the microprojections. Thus, the often expensive agents are coated only on those portions of the device which pierce into the skin, i.e., only the microprojections and not the substrate is coated with the agent.

The present invention provides a device and method for selectively providing multiple applications of an agent formulation on the skin piercing portions of a microprojection member to form an agent-containing coating on the microprojections.

The formulation is dried to form a solid coating which contains the agent.

The microprojections are adapted to pierce through the stratum corneum into the underlying epidermis layer, or epidermis and dermis layers, but, preferably, do not penetrate so deep as to reach the capillary beds and cause significant bleeding. Upon piercing the stratum corneum layer of the skin, the agent-containing coating is dissolved by body fluid (intracellular fluids and extracellular fluids such as interstitial fluid, blood, or mixtures thereof) and released into the skin for local or systemic therapy.

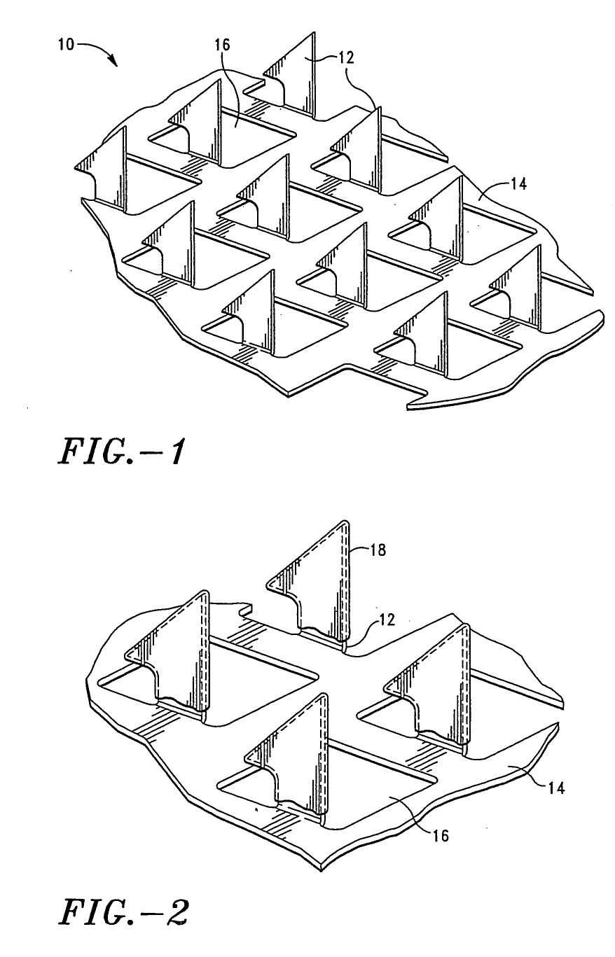

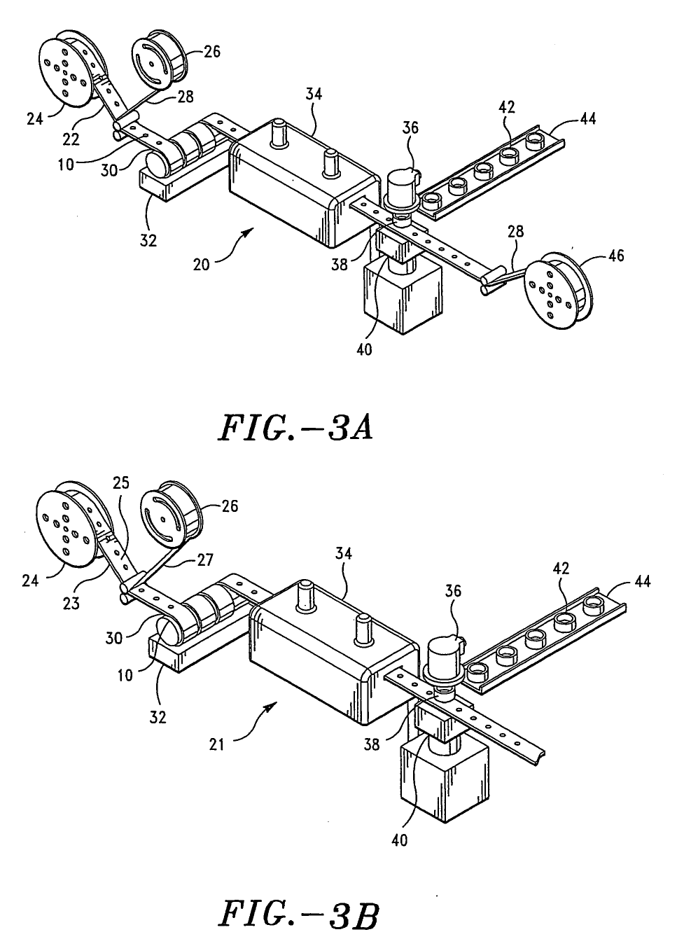

FIG. 1 illustrates one embodiment of stratum corneum-piercing microprojection member 10 for use with the present invention. FIG. 1 shows a portion of the member 10 having a plurality of microprojections 12. The microprojections 12 extend at substantially a 90° angle from a sheet 14 having openings 16. The member 10 may be incorporated in an agent delivery or sampling system including a backing and adhesive for adhering the system to the skin. In the embodiment of the microprojection member 10 shown in FIGS. 1 and 2, the microprojections 12 are formed by etching or punching a plurality of microprojections 12 from a thin metal sheet 14 and bending the microprojections 12 out of a plane of the sheet. Metals such as stainless steel and titanium are preferred. Metal microprojection members and methods of making same are disclosed in Trautman et al, U.S. Pat. No. 6,083,196; Zuck U.S. Pat. No. 6,050,988; and Daddona et al., U.S. Pat. No. 6,091,975 the disclosures of which are incorporated herein by reference. Other microprojection members that can be used with the present invention are formed by etching silicon using silicon chip etching techniques or by molding plastic using etched micro-molds. Silicon and plastic microprojection members are disclosed in Godshall et al. U.S. Pat. No. 5,879,326, the disclosures of which are incorporated herein by reference.

As indicated, the microprojections 12 preferably have a projection length less than approximately 1000 μm. In one embodiment, the microprojections 12 have a projection length of less than 500 μm, more preferably, less than 250 μm. The microprojections 12 also preferably have a width in the range of approximately 25-500 μm and thickness in the range of approximately 10-100 μm.

In further embodiments of the invention, the biocompatibility of the microprojection member 10 can be improved to minimize or eliminate bleeding and irritation following application to the skin of a subject. Specifically, the microprojections 12 can have a length less than 145 μm, more preferably, in the range of approximately 50-145 μm, and even more preferably, in the range of approximately 70-140 μm. Also, the microprojection member 10 comprises an array preferably having a microprojection density greater than 100 microprojections/cm², and more preferably, in the range of approximately 200-3000 microprojections/cm².

Further details regarding microprojection members having improved biocompatibility are set forth in U.S. Application Ser. No. 60/653,675, filed Feb. 15, 2005, which is hereby incorporated by reference in its entirety.

FIG. 2 illustrates the microprojection member 10 having microprojections 12 having a biologically active agent-containing solid coating 18. The coating 18 may partially or completely cover the microprojections 12.

In accordance with the present invention, the agent-containing coating is applied after the microprojections 12 are formed (i.e., etched) and bent out of the plane of metal sheet 14. The coating on the microprojections 12 can be formed by a dip-coating using a reservoir, or more preferably, a roller-coating apparatus as disclosed in U.S. Pat. No. 6,855,372, which is hereby incorporated by reference in its entirety.

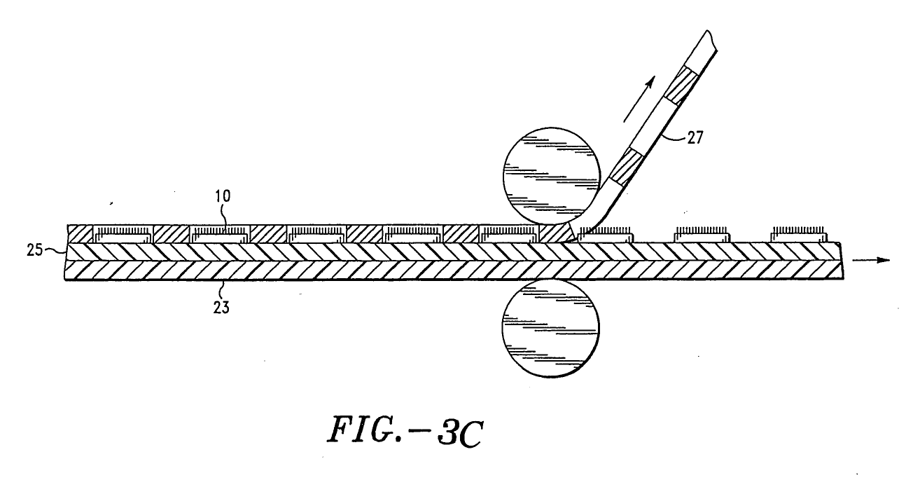

Referring now to FIG. 3A, there is shown one embodiment of a system 20 for providing multiple applications of an agent formulation to coat a microprojection member 10. According to the invention, a continuous, flexible conveyor strip 22 having a series of microprojection members 10 is loaded on product reel 24. Upper reel 26 is designed and positioned to laminate an adhesive backing 28 to strip 22, which then is spiral wrapped around product drum 30 a number of times corresponding to the number of desired coating applications.

The microprojection members 10 are then conveyed through coating reservoir 32, having a formulation of the biologically active agent, to and through a drying chamber 34 for final drying. After drying, the microprojection members 10 with the adhesive backing 28 are die cut at laser cutting station 36 and mounted to a retainer ring 38 by plunger 40. The assembled product 42 is conveyed by belt 44 for further processing, while excess adhesive backing 28 is taken up by outlet reel 46.

Referring now to FIG. 3B, there is shown an alternative embodiment of a system 21 for providing multiple applications of an agent formulation to a microprojection member 10. As illustrated in FIG. 3B, the system 21 is very similar to the system 20 shown in FIG. 3A. However, in this embodiment, the flexible conveyor strip 23 includes an adhesive layer 25 having the series of microprojection members 10 disposed and spaced sequentially thereon. A cross sectional view of the portion of FIG. 3B that shows the flexible conveyor strip 23 with an adhesive layer 25 is illustrated in FIG. 3C.

The conveyor strip 23 further includes a protective layer 27 that is disposed over the adhesive layer 25 and microprojection members 10. Prior to entry into the coating reservoir, the protective layer 27 is removed and taken up by reel 26.

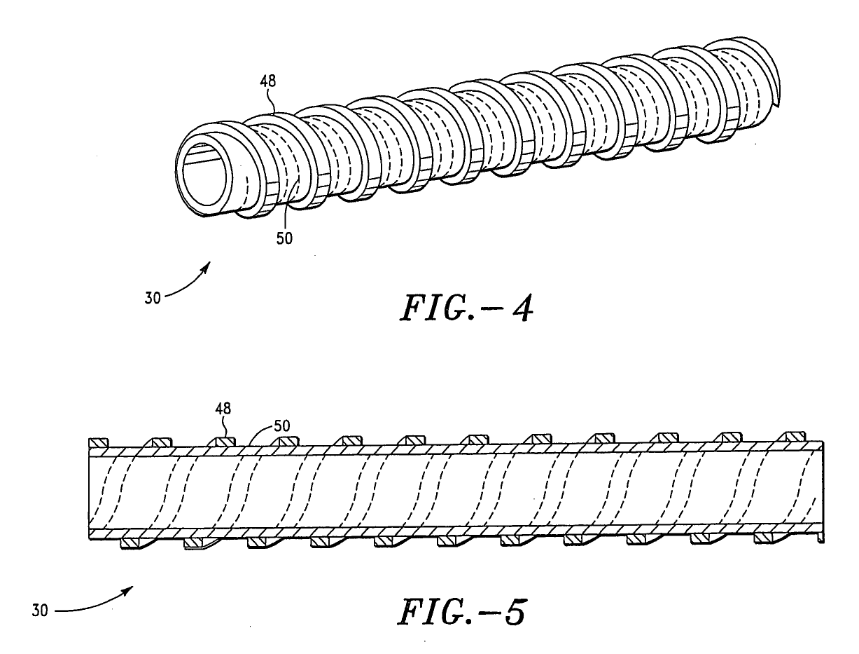

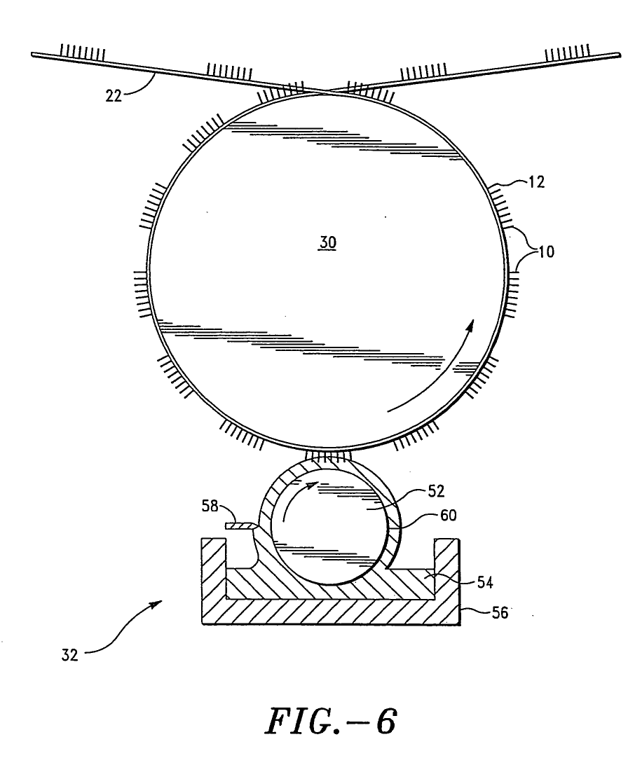

Referring now to FIGS. 4 and 5, a suitable product drum 30 is shown in perspective (FIG. 4) and in cross section (FIG. 5). Product drum 30 utilizes a spiral flight 48 to constrain strip 22 in the appropriate spiral wrap configuration. Preferably, product drum 30 is fixed and comprises a plurality of holes 50 adapted to provide air bearing when compressed air is supplied to the interior of drum 30. This minimizes friction between strip 22 and product drum 30 to allow smooth travel. Alternatively, product drum 30 can be configured to rotate with the movement of strip 22.

Referring to FIG. 6, there is shown a side view of product drum 30, illustrating the interaction of strip 22 of microprojection members 10 as they are coated in reservoir 32. Specifically, coating reservoir 32 includes a rotating coating drum 52 partially immersed in a bath of agent formulation 54. The agent formulation 54 is contained within open container 56. Coating drum 52 rotates in a clockwise manner as shown by the arrow. Coating drum 52 has a surface area sufficient to interact with each wrap of strip 22 as it is conveyed past reservoir 32. The formulation 54 is picked up by the surface of the rotating drum 52 as it rotates through the bath. A doctor blade 58 is provided to control the thickness of the agent formulation downstream from the doctor blade 58. Thus, the outer surface 60 of the rotating drum 52 acts as an agent formulation holding surface to create a region for transferring agent formulation 54 to microprojections 12 of the microprojection members 10 that are being conveyed by strip 22 in the counterclockwise direction shown.

As strip 22 moves around product drum 30, it enters a “coating zone” at the bottom of the drum 30, or between about the 7 o'clock and 5 o'clock positions on the circumference. As can be appreciated, microprojections 12 dip most deeply into the agent formulation at the 6 o'clock position. Preferably, microprojections 12 are dipped to a depth less than the microprojection length. Once microprojection member 10 travels from about the 5 o'clock position around the drum 30 to about the 7 o'clock position on the next wrap, microprojection member 10 is subjected to air drying, forming a portion of solid coating 18 on microprojections 12. Each microprojection member 10 is dipped in agent formulation 54 a number of times corresponding to the number of wraps of strip 22 around product drum 30.

As will be appreciated by one having ordinary skill in the art, the use of a spiral wrap around product drum 30 provides multiple dips to coat a microprojection member 10 from a single reservoir 32, yet utilizes a continuous strip process. By employing a single reservoir, “hold-up” volume is minimized to correspondingly maximize the percentage yield of the process. Further, system 20 is a continuous strip process capable of generating a high volume of coated microprojection members. Given that biologically active agents are often quite expensive, the higher yield offered by this process is very beneficial. Also, commercial aseptic production typically requires the use of isolators, making the compact, space efficient arrangement of a single coating reservoir very attractive. Further, the spiral product drum 30 allows flexibility to vary the number of dips simply by varying the number of wraps.

Although the invention has been described in reference to a preferred embodiment employing a roller coating device, one having skill in the art will recognize that other means for applying an agent formulation to a microprojection member. In general, the spiral wrap of strip 22 around product drum 30 sequentially presents each microprojection member 10 to a location on drum 30 where the formulation is applied. For example, a tank having a controlled level of agent formulation can be used. Other coating mechanisms such as disclosed in U.S. patent application Ser. Nos. 10/608,304, 10/880,701 and 10/984,510, which are hereby incorporated by reference in their entirety.

The agent formulations used in the present invention are typically solutions or suspensions of the biologically or pharmacologically active agent, most typically aqueous solutions or suspensions. The agent formulations preferably have a viscosity in the range of approximately 5 to 500 centipoise (cP), and more preferably in the range of approximately 20 to 50 cP, when measured at a temperature of 25° C. and a shear strain rate of 100 sec⁻¹, in order to effectively coat the tiny stratum corneum-piercing elements to an appropriate thickness. Since multiple applications of agent formulation are employed, the formulation can have relatively less viscosity to facilitate a more uniform application.

The desired thickness of the solid coating on the microprojections is dependent upon the density of the microprojections per unit area and the viscosity and concentration of the coating composition as well as the coating method chosen. In general, the coating thickness is preferably less than 50 μm since thicker coatings have a tendency to slough off the microprojections upon stratum corneum piercing. Preferred coating thicknesses are less than 10 μm as measured from the microprojection surface. Generally, coating thickness is referred to as an average coating thickness measured over the coated microprojection. More preferred coating thicknesses are in the range of approximately 0.1 to 10 μm.

The kinetics of the agent-containing coating dissolution and release will depend on many factors including the nature of the agent, the coating process, the coating thickness and the coating composition (e.g., the presence of coating formulation additives). Depending on the release kinetics profile, it may be necessary to maintain the coated microprojections in piercing relation with the skin for extended periods of time (e.g., up to about 8 hours). This can be accomplished by anchoring the microprojection member to the skin using adhesives or by using anchored microprojections such as described in WO 97/48440, incorporated by reference in its entirety.

The apparatus and method of the present invention have particular utility with coating high potency agents requiring a dose of about 1 mg or less, preferably about 0.25 mg or less. Amounts within this range can be coated onto a microprojection array of the type shown in FIG. 1 having the sheet 14 with an area of up to 10 cm² and a microprojection density of up to 500 microprojections per cm² of the sheet.

In one embodiment of the invention, the coating comprises a formulation having a biologically active agent selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl] carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

In another embodiment of the invention, the biologically active agent comprises a formulation having at least one immunologically active agent, including, but not limited to, viruses and bacteria, protein-based vaccines, polysaccharide-based vaccine, and nucleic acid-based vaccines.

Suitable antigenic agents that can be used in the present invention include, without limitation, antigens in the form of proteins, polysaccharide conjugates, oligosaccharides, and lipoproteins. These subunit vaccines in include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxine subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant - expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitidis (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as respiratory syncytial virus (RSV), cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitidis, pseudomonas aeruginosa, streptococcus pneumoniae, treponema pallidum, and vibrio cholerae, and mixtures thereof.

A number of commercially available vaccines, which contain antigenic agents, also have utility with the present invention including, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Immunologically active agents comprising nucleic acids that can be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants, which, together with the antigenic agent, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β-1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (IL-1β 1, 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL 10, gamma interferon, and NF kappa B regulatory signaling proteins can be used. The noted immunologically active agents can also be in various forms, such as free bases, acids, charged or uncharged molecules, components of molecular complexes or pharmaceutically acceptable salts. Further, simple derivatives of the agents (such as ethers, esters, amides, etc.), which are easily hydrolyzed at body pH, enzymes, etc., can be employed.

Further suitable antigenic agents include antigens in the form of proteins, polysaccharide conjugates, oligosaccharides, lipoproteins, subunit vaccines, Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant - expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

In all cases, following coating, the agent formulation is dried onto the microprojections by various means. In one embodiment the coated device is dried in ambient room conditions. In other embodiments, an oven may be used to provide final drying after the multiple applications of agent formulation have been transferred to the microprojections. Various temperatures and humidity levels can be used to dry the coating solution onto the microprojections. Additionally, the devices can be heated, lyophilized, freeze dried or similar techniques used to remove the water from the coating.

Other known formulation adjuvants can be added to the coating solution as long as they do not adversely affect the necessary solubility and viscosity characteristics of the coating solution and the physical integrity of the dried coating.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A device for applying a biologically active agent formulation to a microprojection member having a plurality of stratum corneum-piercing microprojections, comprising a flexible conveyor strip having said microprojection member mounted thereon, a product drum, wherein said conveyor strip is spiral wrapped around said product drum, and a reservoir of said biologically active agent formulation adapted to cooperate with said product drum to transfer at least one application of said biologically active agent onto said microprojections as said conveyor strip travels around said product drum.

2. The device of claim 1, wherein at least two applications of said biologically active agent are transferred to said microprojections.

3. The device of claim 1, wherein said product drum rotates with said flexible conveyor strip.

4. The device of claim 1, wherein said product drum is fixed.

5. The device of claim 4, wherein said product drum further includes a spiral flight configured to constrain said conveyor strip in a spiral wrap pattern.

6. The device of claim 5, wherein said product drum further includes a plurality of air holes on a surface of said product drum between said spiral flight, wherein said air holes are configured to transmit compressed air to reduce friction between said conveyor strip and said product drum.

7. The device of claim 1, wherein said reservoir comprises an agent formulation holding surface and wherein said product drum is positioned relative to said holding surface so that said microprojection member is conveyed with said microprojections being immersed at a predetermined level through said agent formulation on said holding surface.

8. The device of claim 6, wherein said agent formulation holding surface comprises an outer surface of a rotatable cylindrically-shaped coating drum adapted to receive a film of said biologically active agent formulation.

9. The device of claim 7, wherein said coating drum is partially immersed in a container of said biologically active agent formulation.

10. The device of claim 8, wherein said coating drum has a surface area sufficient to interact with each wrap of said conveyor strip at a desired location on said product drum.

11. A system for applying a biologically active agent formulation to a microprojection member having a plurality of stratum corneum-piercing microprojections comprising:
a flexible conveyor strip having said microprojection member mounted thereon;
a product drum, wherein said conveyor strip is spiral wrapped around said product drum; and
a reservoir of said biologically active agent formulation adapted to cooperate with said product drum to transfer at least one application of said biologically active agent onto said microprojections as said conveyor strip travels around said product drum;
wherein said conveyor strip has a plurality of said microprojection members mounted thereon.

12. The system of claim 11, wherein at least two applications of said biologically active agent are transferred to said microprojections.

13. The system of claim 11, further including a drying chamber adapted to receive said conveyor strip carrying said microprojection members after transfer of said biologically active agent formulation to said microprojections.

14. The system of claim 11, further including a source of adhesive backing for laminating to said microprojection members.

15. The system of claim 11, further including a cutter to cut individual microprojection members from said conveyor strip.

16. The system of claim 15, further including a plunger for mounting said individual microprojection members on a retainer ring.

17. A method for applying a biologically active agent formulation to a microprojection member having a plurality of stratum corneum-piercing microprojections, comprising the steps of:
providing a flexible conveyor strip having said microprojection member mounted thereon;
spiral wrapping said conveyor strip around a product drum; and
conveying said microprojection member past a reservoir of said biologically active agent formulation to transfer at least one application of said biologically active agent onto said microprojections as said conveyor strip travels around said product drum.

18. The method of claim 17, wherein at least two applications of said biologically active agent are transferred to said microprojections.

19. The method of claim 17, wherein said product drum further includes a spiral flight configured to constrain said conveyor strip in a spiral wrap pattern.

20. The method of claim 19, wherein said product drum further includes a plurality of air holes on a surface of said product drum between said spiral flight, further comprising the step of delivering compressed air through said air holes to reduce friction between said conveyor strip and said product drum.

21. The method of claim 17, wherein said reservoir further comprises a biologically active agent formulation holding surface positioned relative to said product drum so that said microprojection member is conveyed past said reservoir with said microprojections being immersed at a predetermined level through said agent formulation on said holding surface.

22. The method of claim 21, wherein said biologically active agent formulation holding surface comprises a cylindrically-shaped coating drum, further comprising the steps of applying a film of said biologically active agent formulation to said holding surface and rotating said coating drum.

23. The method of claim 22, wherein said step of applying a film of said biologically active agent formulation to said holding surface comprises rotating said coating drum while said coating drum is partially immersed in a container of said biologically active agent formulation.

24. The method of claim 17, further including the step of applying a coating of biologically active agent to multiple microprojection members by providing said conveyor strip with a plurality of said microprojection members mounted thereon.

25. A system for applying a biologically active agent formulation to a microprojection member having a plurality of stratum corneum-piercing microprojections comprising:
a flexible conveyor strip having an adhesive layer and a substantially continuous protective layer removably disposed thereon, said microprojection member being attached to said adhesive layer between said protective layer and said adhesive layer;
a product drum, wherein said conveyor strip is spiral wrapped around said product drum; and
a reservoir of said biologically active agent formulation adapted to cooperate with said product drum to transfer at least one application of said biologically active agent onto said microprojections after said protective layer is removed from said conveyor strip and as said conveyor strip travels around said product drum.

26. The system of claim 25, wherein said conveyor strip has a plurality of said microprojection members mounted thereon.

27. The system of claim 25, wherein at least two applications of said biologically active agent are transferred to said microprojections.

28. The system of claim 26, further including a drying chamber adapted to receive said conveyor strip carrying said microprojection members after transfer of said biologically active agent formulation to said microprojections.

29. The system of claim 26, further including a cutter to cut individual microprojection members from said conveyor strip.

30. A method for applying a biologically active agent formulation to a microprojection member having a plurality of stratum corneum-piercing microprojections, comprising the steps of:
providing a flexible conveyor strip having an adhesive layer and a substantially continuous protective layer removably disposed thereon, said microprojection member being disposed on said adhesive layer between said protective layer and said adhesive layer;
removing said protective layer from said conveyor strip;
spiral wrapping said conveyor strip around a product drum; and
conveying said microprojection member past a reservoir of said biologically active agent formulation to transfer at least one application of said biologically active agent onto said microprojections as said conveyor strip travels around said product drum.

31. The method of claim 30, wherein at least two applications of said biologically active agent are transferred to said microprojections.

32. The method of claim 30, wherein said product drum further includes a spiral flight configured to constrain said conveyor strip in a spiral wrap pattern.

33. The method of claim 32, wherein said product drum further includes a plurality of air holes on a surface of said product drum between said spiral flight, further comprising the step of delivering compressed air through said air holes to reduce friction between said conveyor strip and said product drum.

34. The method of claim 30, wherein said reservoir further comprises a biologically active agent formulation holding surface positioned relative to said product drum so that said microprojection member is conveyed past said reservoir with said microprojections being immersed at a predetermined level through said agent formulation on said holding surface.

35. The method of claim 34, wherein said biologically active agent formulation holding surface comprises a cylindrically-shaped coating drum, further comprising the steps of applying a film of said biologically active agent formulation to said holding surface and rotating said coating drum.

36. The method of claim 35, wherein said step of applying a film of said biologically active agent formulation to said holding surface comprises rotating said coating drum while said coating drum is partially immersed in a container of said biologically active agent formulation.

37. The method of claim 30, further including the step of applying a coating of biologically active agent to multiple microprojection members by providing said conveyor strip with a plurality of said microprojection members mounted thereon.
